(19) United States
(12) Patent Application Publication
(10) Pub. No.: US 2007/0184127 A1
(43) Pub. Date: Aug. 9, 2007

(54) Herbal compositions comprising Portaluca

(76) Inventor: Rulin Xiu, Pahoa, HI (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/657,113

(22) Filed: Jan. 24, 2007

Related U.S. Application Data
(63) Continuation of application No. 10/989,033, filed on Nov. 16, 2004, now abandoned.
(60) Provisional application No. 60/585,857, filed on Jul. 8, 2004.

(51) Int. Cl. A61K 36/254; A61K 36/82; A61K 36/48

(52) U.S. Cl. 424/725; 424/728; 424/729; 424/757

(57) ABSTRACT

The present invention relates to herbal compositions and their use in suppressing appetite, promoting body weight loss, stimulating thermogenesis, and/or treating mood disorders, such as anxiety or anger. An herbal composition of the present invention can comprise Portulaca oleracea, and optionally, Green tea, Lotus leaf, and/or Ginseng.

This application claims the benefit of U.S. Provisional Application No. 60/585,857, filed Jul. 8, 2004, which is hereby incorporated by reference in its entirety.

### DESCRIPTION OF THE INVENTION

The present invention relates to herbal compositions and their use in suppressing appetite, promoting body weight loss, and/or stimulating thermogenesis. The herbal compositions can also be utilized to treat mood disorders, such as anxiety or anger, as well as other disturbances of the nervous system.

An herbal composition of the present invention can comprise Portulaca oleracea, an herbal plant that has fleshy, reddish stems and thick, succulent oval shaped leaves. It is also known as purslane. Compositions can comprise Portulaca oleracea, or they consist essentially of it, where the Portulaca oleracea is the only active herbal ingredient. Compositions can also comprise other herbal ingredients, especially Green tea (Camilia sinensis), Lotus leaf (e.g., Nelumbo nucifera), and/or Ginseng (Panax ginseng)

The present invention also relates to herbal compositions comprising Portulaca and optionally, one or more of the following components: Green tea, Lotus leaf, and Ginseng. The amounts of each herb can be in an amount that is effective to suppress appetite, promote body weight loss, stimulate thermogenesis, and/or treat mood disorders. The compositions can further comprise carriers, excipients, and other agents which are conventionally utilized in the formation of compositions for dietary, supplemental, and medicinal purposes.

The compositions can comprise any effective amounts of the ingredients, including, e.g., the following effective amounts (by weight of the total composition): Portulaca, e.g., from about 15-100%; Green tea, e.g., from about 15-75%; Lotus leaf, e.g., from about 15-75%; and Ginseng, e.g., from about 0.1-25%, where the amounts are selected to total 100%. A composition can also comprise from about 30-40% by weight of Portulaca oleracea, from about 30-40% by weight of Green tea, from about 20-30% by weight of Lotus leaf, and from about 3-8% by weight of Ginseng, or about 35% by weight of Portulaca oleracea, about 35% by weight of Green tea, about 25% by weight of Lotus leaf, and about 5% by weight of Ginseng.

In addition to the mentioned ingredients, the following herbs can also be included in the formula, e.g., Rhubarb (e.g., Rheum palmatum, Rheum officinale, or Rheum tanguticum Maxim), Hawthorn fruit (Crataegus pinnatifida), and/or Alisma plantago-aquatica. Any amount can be included in the formula, including, e.g., from about 0.1% to about 75%, from about 0.5% to about 50%, from about 0.5% to about 25% by weight.

The amounts of the ingredients, individually or in combination, can be effective to suppress appetite, promote body weight loss, stimulate thermogenesis, and/or treat mood disorders. An “effective amount” indicates that the mass of ingredient or ingredients in the composition is useful to achieve the purpose for which it is administered. An herbal composition can comprise effective amounts of Portulaca oleracea and one or more of the following herbs: Green tea, Lotus leaf, or Ginseng. The latter indicates that each herb is present in an amount which is effective for the stated purpose.

Amounts are selected based on various factors, including age, health, gender, and weight of a patient or animal to be treated, etc. Useful amounts of total composition include, about 0.2-20 grams, 0.2-5 grams, 0.2-3 grams, 0.2-2 grams, 0.2 grams, 0.5 grams, 1 grams, 2 grams, 3 grams, etc., per dosage, one or more times a day, such as twice a day, or three times a day, etc., depending upon the subject's physical condition and the potency of the ingredients included. The ingredients can be combined in “synergistic amounts”. By this, it is meant that the amount of an ingredient present in the composition (and in combination with the other ingredients) is more effective at achieving the desired purpose (e.g., promoting weight loss) than when administered alone or in another herbal composition. Synergy indicates that the ingredients cooperate with each other to achieve an enhanced or superior result than would be achieved if either were administered alone, i.e., the effect is not additive (the sum is greater than the sum of the individual parts).

The present invention relates to methods of suppressing appetite and/or promoting body weight loss in a subject, comprising, e.g., administering to a subject in need thereof a composition comprising an effective amount of any composition, ingredient, or ingredients, as mentioned above.

The phrase “suppressing appetite” indicates that the appetite of a subject is reduced, diminished, decreased, etc., in comparison to the subject's normal appetite. If the subject is a human, the effect can be described as being less hungry, not hungry, more easily satiated, etc., in comparison to the appetite before administration. “Promoting body weight loss” indicates that the compositions and methods are useful to encourage, elicit, cause, produce, etc., reduction in the mass of a subject's body. The loss in mass can be from a tissue, such as fat and muscle, where stored lipids, proteins, carbohydrates, and/or liquids, such as water, are lost. The invention is not limited by how much weight loss is promoted of facilitated. For instance, it can be 0.25%, 0.5%, 1%, 5%, 7%, 10%, or more of total body weight. The present invention is not bound by any mechanism through which weight loss is achieved. For example, the weight loss can result from a reduction in the appetite of the subject that is associated with a concomitant decline in the total caloric intake.

In addition, the present invention can relates to methods of reducing total caloric intake by a subject, comprising administering effective amounts of compositions of the present invention to the subject. The reduction in total caloric intake indicates that subject is consuming fewer calories than before administration of the compositions. This can be shown or determined by comparing dietary records (e.g., foods consumed during a day) before taking a composition of the present invention, and afterwards, e.g., after about 2 days, five days, a week, a month, two months, three months, six months, one year, etc.

The present invention relates to methods of regulating mood in a subject, comprising, e.g., administering to a subject in need thereof a composition comprising an effective amount of any composition, ingredient, or ingredients, as mentioned above. By the term “mood” it is meant state of subject's emotion, especially anxiety or anger (including hostility).

The present invention also relates to methods of stimulating thermogenesis in a subject, comprising, e.g., administering to a subject in need thereof a composition comprising an effective amount of any composition, ingredient, or ingredients, as mentioned above. Thermogenesis, or non-shivering heat production, is a metabolic process in which calories consumed by the body are utilized to dissipate energy as heat. It primary occurs in adipocytes (fat cells), such as brown adipose tissue. By stimulating thermogenesis, it is meant that compositions of the present invention promote, induce, etc., non-shivering heat production that causes the fat cells to burn stored energy. A consequence of this can be a loss of body weight.

The desired effect of a composition of the present invention can be achieved after a single dosage, but also after the subject has taken a plurality of dosages. For example, it can take more than one day of administration for the composition to reach its maximum benefit, e.g., about or more than one week, about or more than two weeks, about or more than one month, about or more than six weeks, about or more than two mronths, etc.

To achieve the above-mentioned effects, a composition in accordance with the present invention is administered to a subject. By the term “administered,” it is meant that the composition is delivered to a subject by any means or route which is effective to achieve the desired result, including, e.g., oral, parenteral, enteral, intraperitoneal, topical, transdermal (e.g., using any standard patch), ophthalmic, nasally, local, non-oral, such as aerosal, inhalation, subcutaneous, intravenous, intramuscular, buccal, sublingual, rectal, vaginal, intra-arterial, intrathecal. A subject can be any individual, human or non-human animal (e.g., cow, pig, sheep, goat, mice, rats, pets, such as dogs and cats, etc.) in need of weight modulation.

Compositions can be administered at any time suitable time. For example, compositions can be administered, e.g., prior or after a meal, along with a meal, prior to exercise, prior to a sporting event, prior to bedtime, etc. Where weight-loss is desired, compositions of the invention can be administered when nutrients from a meal or snack begin to appear in the blood, i.e., the absorptive state, when ingested nutrients are entering the blood from the gastrointestinal tract. See, e.g., Human Physiology, Vander et al., Fifth Edition, 1990, e.g., Chapters 16 and 17. The adsorptive state can vary, depending upon the content of the food, e.g., from about 1 hour to about 4 hours.

The compositions can be in any effective form, including, oral, pill, capsule, troche, liquid, gel, powder, extract, beverage, shake, food, tea, topical, injectable, etc. They can be administered alone, or in combination with other active or inert agent(s).

Any effective part of an herb in accordance with the present invention can be used, including to seeds, leaves, stems, flowers, roots, berries, bark, or any other plant parts that are useful for the purposes described. The effective parts of Portulaca are generally the stems and leaves, and also seeds. Herbs can be in any form which is effective, including, but not limited to dry powders, grounds, emulsions, extracts, and other conventional compositions. To extract or concentrate the effective ingredients of an herb, typically the plant part is contacted with a suitable solvent, such as water, alcohol, methanol, or any other solvents, or mixed solvents. The choice of the solvent can be made routinely, e.g., based on the properties of the active ingredient that is to be extracted or concentrated by the solvent.

Herbs can be used as 5:1 extract, e.g., an aqueous extract. This indicates that the dry weight of the herb is concentrated five times (5:1) in the extract. Any of the aforementioned formula can be prepared with a 5:1 extract, and any of the aforementioned values can refer to the weight of the extract.

Green tea can be used as an extract containing about 10% caffeine and about 18% epigallocatechin) or 5:1 extract prepared from a dry powder. It can also be used as an extract containing about 7% caffeine and about 25% polyphenol. Portulaca can be used as a dry powder or 5:1 to 20:1 extract prepared from a dry powder or freshly picked wet herb. Ginseng can be used as dry root powder, or a leaf or root extract containing, e.g., about 5-60% Ginsenoid or 5:1 to 20:1 extract prepared from a dry root or leaf powder. Lotus leaf can be used as dry powder or a 5:1 to 20:1 extract prepared from a dry powder or freshly picked lotus leaf.

Compositions of the present invention can specifically exclude one or more of the following components (or any other ingredients mentioned in this disclosure): imperata rhizome, broadleaf holly leaf, agastache and liquorice root, chitin, batryticated silkworm powder, rhubarb, polygonatum root, lycium berry, gynostemma pentaphyllum, glechoma longituba, mung bean, jujube, haw, wolfberry fruit, lentinus edodes or lucid ganoderma, tangerine peel, honeysuckle flower, white chrysanthemum, and/or carrot.

Compositions can further comprise inert and carrier ingredients, stabilizers, anti-oxidants, etc., including, but not limited to, water, salt solutions, alcohols, gum arabic, vegetable oils, benzyl alcohols, gelatin, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose and the like. Other additives include, e.g., antioxidants and preservatives, coloring, flavoring and diluting agents, emulsifying and suspending agents, such as acacia, agar, alginic acid, sodium alginate, bentonite, carbomer, carrageenan, carboxymethylcellulose, cellulose, cholesterol, gelatin, hydroxyethyl cellulose, hydroxppropyl cellulose, hydroxypropyl methylcellulose, methylcellulose, octoxynol 9, oleyl alcohol, povidone, propylene glycol monostearate, sodium lauryl sulfate, sorbitan esters, stearyl alcohol, tragacanth, xanthan gum, and derivatives thereof, solvents, and miscellaneous ingredients such as microcrystalline cellulose, citric acid, dextrin, dextrose, liquid glucose, lactic acid, lactose, magnesium chloride, potassium metaphosphate, starch, and the like.

Without further elaboration, it is believed that one skilled in the art can, using the preceding description, utilize the present invention to its fullest extent. The entire disclosure of all applications, patents and publications, cited above and in the figures are hereby incorporated by reference in their entirety, including U.S. Provisional Application No. 60/585,857, filed Jul. 8, 2004.

What we claim:

1. An herbal composition comprising effective amounts of Portulaca oleracea and one or more of the following herbs: Green tea, Lotus leaf, or Ginseng, wherein said amounts are effective for suppressing appetite.

2. An herbal composition of claim 1, comprising effective amounts of Portulaca oleracea and Green tea.

3. An herbal composition of claim 1, comprising effective amounts of Portulaca oleracea and Lotus leaf.

4. An herbal composition of claim 1, comprising effective amounts of Portulaca oleracea and ginseng.

5. An herbal composition of claim 2, comprising effective amounts of Portulaca oleracea, Green tea, and Ginseng.

6. An herbal composition of claim 3, comprising effective amounts of Portulaca oleracea, Lotus leaf, and Ginseng.

7. An herbal composition of claim 1, comprising effective amounts of Portulaca oleracea, Green tea, Lotus leaf, and Ginseng.

8. An herbal composition of claim 7, comprising from 20-50% by weight of Portulaca oleracea, from 20-40% by weight of Green tea, from 10-35% by weight of Lotus leaf, and from 0.5-10% by weight of Ginseng.

9. An herbal composition of claim 7, comprising from 30-40% by weight of Portulaca oleracea, from 30-40% by weight of Green tea, from 20-30% by weight of Lotus leaf, and from 3-8% by weight of Ginseng.

10. An herbal composition of claim 1, where said amounts are further effective for promoting body weight loss.

11. An herbal composition of claim 2, wherein the Green tea is an extract comprising 25% polyphenol and 7% caffeine.

12. An herbal composition of claim 1, wherein said amounts are synergistic.

13. A method of suppressing appetite, comprising administering an effective amount of a composition of claim 1.

14. A method of suppressing appetite, comprising administering an effective amount of a composition of claim 7.

15. A method of suppressing appetite, comprising administering a composition consisting essentially of an effective amount of Portulaca oleracea.

16. A method of suppressing appetite, comprising administering a composition comprising an effective amount of Portulaca oleracea.

17. A method of promoting weight loss, comprising administering an effective amount of a composition of claim 1.

18. A method of claim 13, wherein 0.5-3 grams a day of said composition is administered.
